(19) United States
(12) Patent Application Publication
(10) Pub. No.: US 2019/0000758 A1
(43) Pub. Date: Jan. 3, 2019

(54) ANIMAL FEED BOLUS AND METHODS FOR MANUFACTURING THE SAME

(71) Applicant: CAN TECHNOLOGIES, INC., Wayzata, MN (US)

(72) Inventors: Jean Marie BEGUIN, Langueux (FR); Rene-Pol DAGORNE, Planguenoual (FR)

(73) Assignee: CAN TECHNOLOGIES, INC., Wayzata, MN (US)

(21) Appl. No.: 16/067,249

(22) PCT Filed: Nov. 22, 2016
(86) PCT No.: PCT/US16/63279

§ 371 Date: Jun. 29, 2018

Related U.S. Application Data
(60) Provisional application No. 62/276,389, filed on Jan. 8, 2016.

(51) Int. Cl. A61K 9/00; A23K 20/174; A23K 50/10; A23K 20/20; A61K 9/20; A61K 31/375; A61K 31/355; A61K 33/30; A61K 33/04; A61K 38/44

(52) U.S. Cl. CPC A61K 9/0065; A23K 20/174; A23K 50/10; A23K 20/30; A61K 9/2072; C12Y 115/01001; A61K 31/355; A61K 33/30; A61K 33/04; A61K 38/446; A61K 31/375

(57) ABSTRACT

The present invention relates to an animal feed bolus having two or more sections, wherein each section has a different composition. The different sections of the bolus are formulated to have different release times and/or to provide compositions with different nutritional purposes. Also described herein are methods for manufacturing such boluses, and methods for feeding an animal using such boluses.

### CROSS-REFERENCE TO RELATED APPLICATIONS

This application claims the benefit of U.S. Provisional Application No. 62/276,389, filed Jan. 8, 2016, which is hereby incorporated by reference in its entirety.

### BACKGROUND OF THE INVENTION

A bolus is a large pill administered to an animal orally using a special applicator. The composition of the bolus can include minerals, vitamins, and trace elements. The bolus diffuses into the rumen to gradually release its nutrients over time, for example a period of one hour to a period of several months, depending on the formulation and the objective. Each bolus is generally formulated to target a single physiological objective, for example to support calving, peak of lactation, dry period, reproduction, or grazing period in cows. However, currently available boluses are formulated with only a single homogenous composition, and a single bolus generally cannot be used to target multiple physiological objectives.

### SUMMARY OF THE INVENTION

Described herein are animal feed boluses having two or more sections, wherein each section has a different formulation or composition. The different sections can be modified to have different release times and/or to provide compositions with different nutritional purposes. The boluses are preferably used for providing a complementary feed composition for an animal. Also described herein are methods for manufacturing such boluses, and methods for feeding an animal using such boluses.

In one aspect, an animal feed bolus is described, comprising: a first section, and a second section connected to the first section, wherein the first section has a different composition and release rate than the second section. In some embodiments, the release rate of the first section is less than 15 days. In some embodiments, the release rate of the first section is less than one week. In some embodiments, the release rate of the first section is less than one day. In some embodiments, the release rate of the first section is less than 12 hours, less than 6 hours, or less than 1 hour.

The second section preferably has a slower release rate than the first section. In some embodiments, the release rate of the second section is greater than 2 days. In some embodiments, the release rate of the second section is greater than 1 week. In some embodiments, the release rate of the second section is greater than 30 days. In some embodiments, the release rate of the second section is greater than 6 months.

In one aspect, the size and/or weight of the respective sections can vary. In some embodiments, the first section is about 50 wt % of the bolus. In some embodiments, the first section is about 40 wt % of the bolus. In other embodiments, the first section is less than about 30 wt % of the bolus; less than about 20 wt % of the bolus, or less than about 10 wt % of the bolus.

In one aspect, the compositions of the various sections of the bolus are different. In some embodiments, the first section or any other section with a relatively fast-release time comprises vitamins A, D3, E, B1, B3, B12, or C, or any combination thereof. In some embodiments, the other section(s) of the bolus can also include vitamins A, D3, E, B1, B3, B12, or C, or any combination thereof. In some embodiments, the second section or any other section with a relatively slow-release time comprises a nutrient selected from the group consisting of zinc, selenium, manganese, copper, iodine, and cobalt, sulfates or any other salts thereof, oxides thereof, organic forms thereof, and any combinations thereof. In some embodiments, the second section or any other section with a relatively slow-release time comprises one or more aromatic extracts.

In one aspect, the first section and/or second section can contain any vitamin, aromatic extract, trace mineral, or any other material suitable for an animal feed. In one aspect, the second section can include some or all of the same materials as the first section.

In some embodiments, the first and section sections are connected via a binder. In some embodiments, the bolus can include a third section connected to the first and/or second sections. In one such embodiment, the third section has a different release rate than the first and/or second sections.

In one aspect, a method of manufacturing an animal feed bolus is described, comprising: providing a first pre-mix powder, compressing the first pre-mix powder into a first bolus section, providing a second pre-mix powder, and compressing the second pre-mix powder into a second bolus section to form an animal feed bolus, wherein the first and second bolus sections are connected. In one aspect, a method of manufacturing an animal feed bolus is described, comprising: forming a first bolus section by solidifying a first pre-mix paste or liquid, and forming a second bolus section by solidifying a second pre-mix paste or liquid, wherein the first bolus section and second bolus section are connected. In some embodiments, the first and second bolus sections have different release rates. In one aspect, the method of manufacturing an animal feed bolus is used to manufacture any embodiment of the bolus described herein.

In one aspect, a method for feeding an animal is described, comprising: providing a bolus having a first section and a second section, wherein the first section is connected to the second section and the first and second sections have different release rates, and administering the bolus to the animal. In one aspect, the animal is a ruminant. In one aspect, the bolus remains in the reticulum of the ruminant after administration. In one aspect, the method for feeding an animal can include feeding an animal with any embodiment of the boluses described herein. In some embodiments, the method includes administering more than one bolus to the animal. In one such embodiment, the boluses can have the same composition and/or types of sections. In other embodiments, the boluses can have different compositions and/or types of sections.

### BRIEF DESCRIPTION OF THE DRAWINGS

The following detailed description of the invention will be better understood when read in conjunction with the appended drawings. It should be understood, however, that the invention is not limited to the precise arrangements and instrumentalities of the embodiments shown in the drawings.

FIG. 1 is a diagram of an exemplary embodiment of an animal feed bolus having multiple sections.

FIG. 2 is a graph and corresponding set of images showing the release of materials from an exemplary embodiment of a bolus in an artificial saliva solution over time at controlled temperature and pH. The materials in the first section of the bolus, i.e., the top section, are released significantly faster than the materials in the second section.

FIG. 3 is a graph showing the release of trace minerals and vitamins from an exemplary embodiment of a bolus in the reticulo rumen of three different cattle.

FIG. 4, comprising FIGS. 4A and 4B, is a set of graphs showing the evolution plasmatic vitamin E (FIG. 4A) and vitamin C (FIG. 4B) concentrations in cattle during application of exemplary boluses of the present invention (each cow receiving two boluses).

FIG. 5 is a graph showing the evolution of plasmatic vitamin E before (day 0) and after application (day 30) of an exemplary bolus of the present invention.

FIG. 6 is a graph showing somatic cells count (SCC) in cows that were administered exemplary boluses of the present invention compared to SCC in cows not administered such boluses.

FIG. 7 is a graph showing milk production in cows that were administered exemplary boluses of the present invention compared to milk production in cows not administered such boluses.

### DETAILED DESCRIPTION

It is to be understood that the figures and descriptions of the present invention provided herein have been simplified to illustrate elements that are relevant for a clear understanding of the present invention, while eliminating other elements found in the related field(s) of art. Those of ordinary skill in the art would recognize that other elements or steps may be desirable or required in implementing the present invention. However, because such elements or steps are well known in the art or do not facilitate a better understanding of the present invention, a discussion of such elements or steps is not provided herein.

Unless defined otherwise, all technical and scientific terms used herein have the same meaning as commonly understood by one of ordinary skill in the art to which this invention belongs. As used herein, each of the following terms has the meaning associated with it as defined in this section.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 7 should be considered to have specifically disclosed subranges such as from 1 to 3, from 1 to 4, from 1 to 6, from 2 to 5, from 3 to 5, etc., as well as individual numbers within that range, for example, 1, 2, 3, 3.6, 4, 5, 5.8, 6, 7, and any whole and partial increments in between. This applies regardless of the breadth of the range.

Animal Feed Bolus

The present invention relates to an animal feed bolus that is particularly useful for feeding ruminants. In some embodiments, the bolus includes a first composition that releases relatively quickly in an animal's digestive tract, and a second composition that releases more slowly in the digestive tract. In a preferred embodiment, the bolus provides a feed composition that is complementary or supplementary to the animal's regular diet.

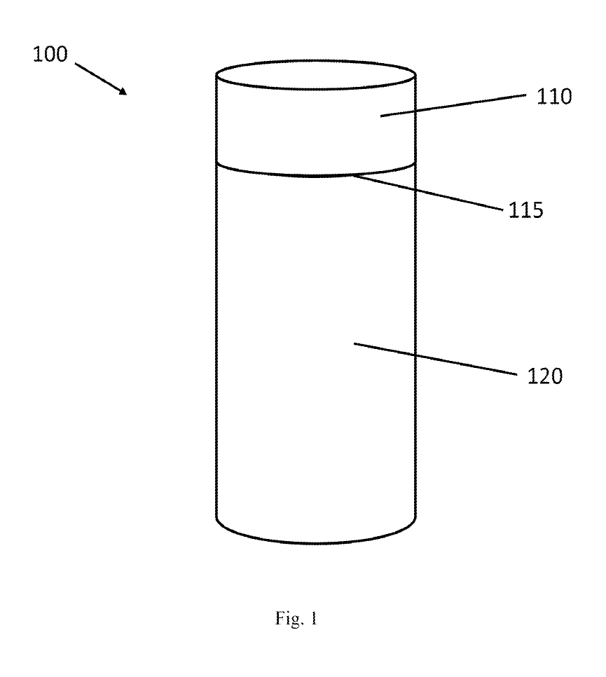

Referring now to FIG. 1, an exemplary embodiment of an animal feed bolus 100 is shown. Bolus 100 is generally cylindrical in shape and includes a first section 110 and a second section 120. First section 110 and second section 120 are connected together at interface 115. First section 110 and second section 120 are composed of animal feed formulations having different release rates. For example, first section 110 can have a release rate on the order of hours, such as 6 hours, while second section 120 can have a release rate on the order of days, such as 30 days. Accordingly, this dual-release rate bolus can be used to deliver dietary materials at different dosages or rates without the need for administering multiple boluses to the animal.

It is contemplated herein that a person skilled in the art could formulate the sections to have varying release rates from 30 min or less up to 8 months or more. Accordingly, a faster-release section or portion can be used to administer certain dietary components that should be absorbed by the animal relatively quickly and/or at a relatively high dose, while a slower-release section or portion can be used to administer certain dietary components that should be absorbed by the animal at a relatively low dose and/or that should be absorbed by the animal at a consistent dose over a long period of time. Exemplary release times for dietary materials useful for animal feeding can be 30 min, 1 h, 2 h, 4 h, 8 h, 12 h, 18 h, 1 day, 2 days, 3 days, 7 days, 14 days, 28 days, 2 months, 3 months, 6 months, 9 months, or 12 months. However, the release times of the different sections are not limited to any specific release time recited herein, and any bolus section can have any release time, as would be understood by a person skilled in the art. In a preferred embodiment, first section 110 will have a significantly different release rate than second section 120.

The dietary materials useful in the bolus of the present invention include, but are not limited to vitamins, minerals, protein, fiber, amino acids, fats, carbohydrates, and any other additives or feed materials useful for animal nutrition. For example, vitamins can include vitamins A, D3, E, B1, B3, B12, E, and C; minerals can include oxides, sulfates or any other type of salt, and chelates or any other organic form of metals such as zinc, copper, manganese, iodine, cobalt and selenium; and the additives can include various aromatic extracts. However, the boluses of the present invention are not meant to be limited to the specific dietary components listed herein. As would be understood by a person skilled in the art, the boluses can include any dietary components or mixture of such components that would be useful for feeding the target animal. Further, the dietary components formulated in the bolus can be adapted for any purpose, for example to supplement or complement the animal's diet to improve the health, size or weight, or performance of the animal, or to treat, prevent, or reduce the risk of a disease or undesirable condition of the animal, for example to reduce the risk of metabolic disease.

Bolus 100 can also include other materials aside from animal dietary components. The bolus can include materials or additives useful for controlling the release rate of the dietary materials from the bolus sections. Such materials or additives can include, but are not limited to magnesium oxide and magnesium phosphate. The bolus can also include preservatives and/or excipients.

In one embodiment, interface 115 can include a binder to improve or maintain the connection between sections 110 and 120. However, a binder is not required for interface 115, and sections 110 and 120 can be connected by compressing materials together to form bolus 100, or by depositing the material of one section onto the other section. In one embodiment, section 110 and/or 120 can be coated, for example to change the release rate, delay the start of the release of dietary materials, or to prevent the release of materials until the bolus reaches a portion of the digestive tract having a specific pH.

As described herein, each section preferably has a different composition. In one embodiment, each section can include different dietary components that are formulated to be released at different rates. In another embodiment, each section can have at least one dietary component in common, but the common components can be formulated to be released at different rates. Such an embodiment can be used to provide a rapid release of a component to obtain an initial blood concentration in the animal while also providing a slow release of the same component to maintain a desired blood concentration over a longer period of time. In some embodiments, the different sections of the bolus can have some or all of the same components in each section. In some such embodiments, any of the components can be present in different concentrations in the respective sections, e.g., one section can include a relatively small amount of a component for quick release and another section can include a relatively large amount of the same component for slower release.

In some embodiments, the different sections of the bolus can have the same size and/or weight. In other embodiments, the sections can have different sizes and/or weights. In such embodiments, the different sections can be sized to provide a desired target concentration of the respective components in the animal. For example, the quick-release section can be significantly smaller than the slow-release section. In embodiments of the bolus having two sections, the first section can be about 50 wt %, about 40 wt %, about 30 wt %, about 20 wt %, about 10 wt %, about 5%, or less, of the bolus.

In other embodiments, the bolus of the present invention can have more than two sections, for example three, four, five, or more sections. However, the bolus is not limited to any specific number of sections recited herein. Further, when the bolus has three or more sections, the release rates of some sections can be the same. For example, if the bolus has three sections, two sections can have different compositions, but have the same release rate while the third section can have a different release rate. Accordingly, the sections of the bolus of the present invention can have any combination of compositions, sizes, weights, and/or release rates to achieve a desired dietary result in an animal, as would be understood by a person skilled in the art.

In the embodiment of bolus 100 shown in FIG. 1, sections 110 and 120 are layered so that at least a portion of the surface area of each section is available for digestion or diffusion when bolus 100 is administered to an animal. Accordingly, release of the dietary components contained within each section can start to be released as soon as bolus 100 is administered to the animal. In other embodiments, bolus 100 can be constructed such that first portion 110 can be contained within second portion 120, such that the dietary components contained within first portion 110 are not released until some or all of second portion 120 is digested or released. Further, the shape of the bolus can be a shape other than a cylinder, for example a sphere. In some embodiments, the bolus can be tablet-shaped. As would be understood by a person skilled in the art, the bolus is preferably shaped for suitable administration to an animal, e.g., suitably shaped for oral administration.

The bolus can be any size or weight. In some embodiments, the bolus can include most or all of the nutritional requirements of an animal. In other embodiments, the bolus includes only a portion of the nutritional requirements of an animal. Exemplary weights include, but are not limited to, 10 g, 30 g, 60 g, 95 g, 150 g, 200 g, 500 g, or 1 kg. Exemplary diameters for cylindrical boluses can include, but are not limited to 10 mm, 15 mm, 17 mm, 20 mm, 25 mm, 27 mm, or 30 mm Exemplary lengths for cylindrical boluses can include, but are not limited to, 10 mm, 20 mm, 35 mm, 60 mm, 80 mm, 150 mm, or 200 mm.

### Manufacturing Method

Currently available boluses have the same composition throughout. Such boluses are generally made by compressing a premix powder having a homogenous composition, or via solidification of a paste or liquid. However, the boluses described herein have two or more sections, each with varying compositions. To manufacture the boluses of the present invention, a different premix powder, paste, or liquid is used for each section of the bolus. The manufacturing equipment used to make the bolus can form distinct sections by compressing and/or depositing different premixes to the same bolus in succession. In some embodiments, the resulting bolus has different layers corresponding to each unique premix, for example as shown in FIG. 1.

In some embodiments, the method of the present invention includes steps for preparing two or more premix powders, pastes, or liquids. For example, dietary ingredients and other additives are weighed out in the desired amounts and proportions, and then added to a container. The ingredients are then mixed using suitable mixing or blending equipment to form a premix powder, paste, or liquid. The premix powder, paste, or liquid is preferably well-blended and homogenous. The premix powder, paste, or liquid can be transferred to other containers or packaging prior to manufacturing the bolus.

In one embodiment, to manufacture a bolus a first premix powder is added to a press, which compresses the powder to form a first bolus section. A second premix powder is then added to the press, on top of the first bolus section. The second premix powder is then compressed onto the first bolus section to form a single bolus having a first section and a second section. These steps can be repeated to form one or more additional bolus sections connected to the same bolus. The bolus formation can also include the addition of a binding powder between the bolus sections to enhance binding of the different bolus sections to each other. After all sections of the bolus are formed, the bolus is removed from the press and optionally finished to ensure all edges are sufficiently smooth. In some embodiments, the bolus can be coated with other materials, as would be understood by a person skilled in the art. In some embodiments, water or some other liquid can be added to the premix powder at any point of the manufacturing process to facilitate formation of the bolus. In some embodiments, one or more sections of the bolus can be formed by solidifying or drying premix pastes or liquids instead of by compressing premix powders, as would be understood by a person skilled in the art. In one such embodiment, a bolus section formed by solidifying or drying a premix paste or liquid can retain a significant amount of moisture, i.e., the section does not need to be completely dried or solidified to form a bolus section.

In some embodiments, any equipment used for producing single-composition boluses can be adapted or modified to produce the multi-section boluses of the present invention. For example, a second set of equipment suitable for measuring and dispensing a premix powder, paste, or liquid can be added to an existing bolus manufacturing machine, which can be used to manufacture the second section of the boluses.

### Method of Feeding

The bolus of the present invention is particularly useful for feeding ruminants, for example cows, but can be used to feed any type of animal. In one aspect, the bolus is a complementary feed, i.e., the bolus can provide a desired mixture of nutrients that complements the animal's diet. In one embodiment, the bolus provides a dietetic feed, which is a feed for a particular nutritional purpose.

In one embodiment, the bolus is administered to the mouth of a ruminant, where it progresses to the reticulum of the ruminant. In a preferred embodiment, the bolus remains in the reticulum where it releases most or all of the dietary components. As described herein, each portion or section of the bolus can be formulated to provide a different release rate and/or a different composition to address the various dietary needs of an animal.

Boluses of the present invention can preferably be used for feeding cattle. However, other animals, for example sheep or goats can be fed with the boluses described herein. Further, the use of the boluses of the present invention are not limited to ruminants and can include any animal, as would be understood by a person skilled in the art.

In some embodiments, the method of feeding can include administering a bolus to an animal, wherein the bolus includes some feed materials in a fast-release section and some feed materials in a slow-release section. In one aspect, the bolus administered can be any embodiment of an animal feed bolus described herein.

In some embodiments, the method of feeding can include the administration of two or more boluses to an animal. In such embodiments, the multiple boluses can be administered to the animal nearly simultaneously, or can be administered separately at any suitable time interval. As would be understood by a person skilled in the art, the boluses in a multiple-bolus feeding method can have the same composition, or the composition of the boluses can be variable.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

### Example 1: Test of the Quick Release of Vitamin C and Vitamin E

A multi-section animal complementary feed bolus was produced and tested. This test provided an initial evaluation of the release of the bolus before in vivo evaluation.

The bolus included a section for quick-release including vitamin C and vitamin E, and a slow-release section including zinc, selenium, superoxide dismutase (SOD), and aromatic substances.

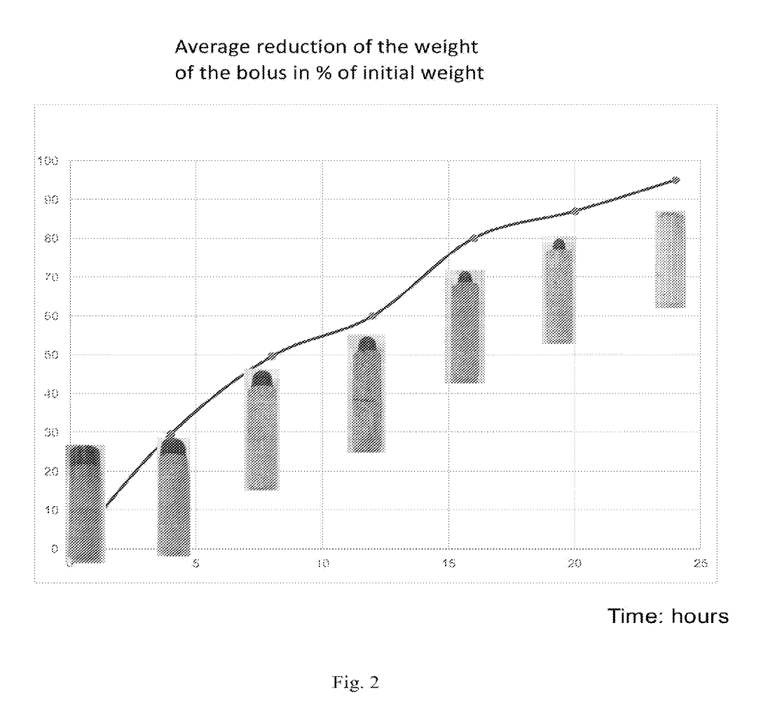

The quick release of the bolus was evaluated in vitro in the laboratory by immersion of the bolus in a solution of artificial saliva at controlled temperature and pH (FIG. 2).

In a beaker, five liters of artificial saliva were prepared at controlled temperature (˜39° C.) and pH (˜6.5). Then the bolus was weighed and introduced into the solution while the solution was continuously agitated. The bolus was weighed at hourly or daily time intervals, and the daily release was reported.

The test shows the quick release (˜6 h) of the smaller section of the bolus.

### Example 2: Test of Bolus Material Release on Fistulated Dairy Cows

An exemplary embodiment of an intra-ruminal sustained-release bolus was tested in order to determine its release rate. A trial was carried out on three rumen-fistulated dairy cows (from Cargill Innovation Center, Velddriel 5334 LD Velddriel, The Netherlands).

All cows were fed on a diet of maize and grass silage, supplemented with soy and rapeseed meal and a commercial dairy concentrate. Each cow was given one bolus, which was introduced in the rumen by way of a rumen fistula, with expected migration into the reticulum. Boluses were retrieved (by hand) after one day and two days incubation, and then twice a week. Retrieved boluses were then brushed clean of extraneous particulate matter, and dried with a paper towel before being weighed and returned to the rumen. Each bolus was weighed and measured. Reduction in size and weight was determined in percentage of the original size and weight of the bolus.

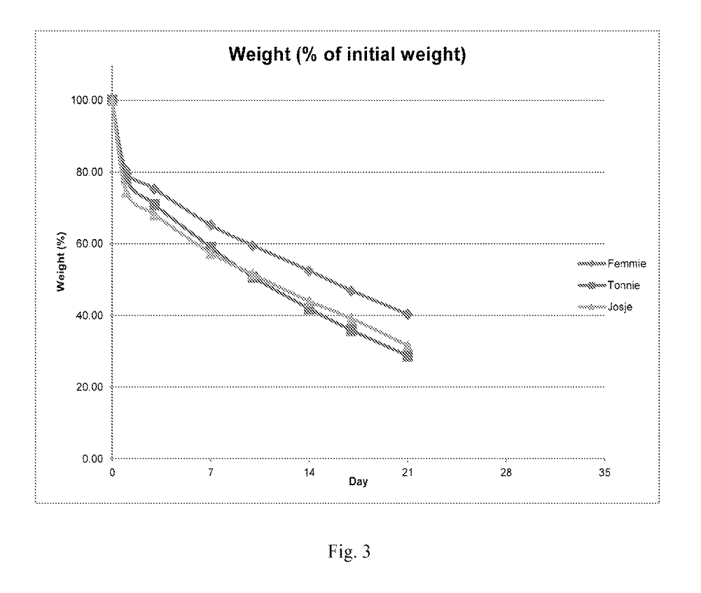

Referring to FIG. 3, the results of this experiment show that the bolus includes a quick-release section which represents approximatively 20% of the bolus and diffuses in less than one day; and a slower-release section which diffuses in 30-35 days.

### Example 3: Test of Efficacy on Dairy Cows

Holstein dairy cows (n=15) from a farm in Western France, were randomly assigned to 3 groups. The cows of each group received two of the boluses according to the present invention at 8 hour intervals. Blood samples were taken before the application of the first bolus, at 9 hours after the application of the first bolus, at the time of the administration of the second bolus (t=17 h), and at 9 hours after the administration of the second bolus (t=25 h). Blood serum was analyzed for vitamin E and vitamin C at the Frank Duncombe's Lab, France.

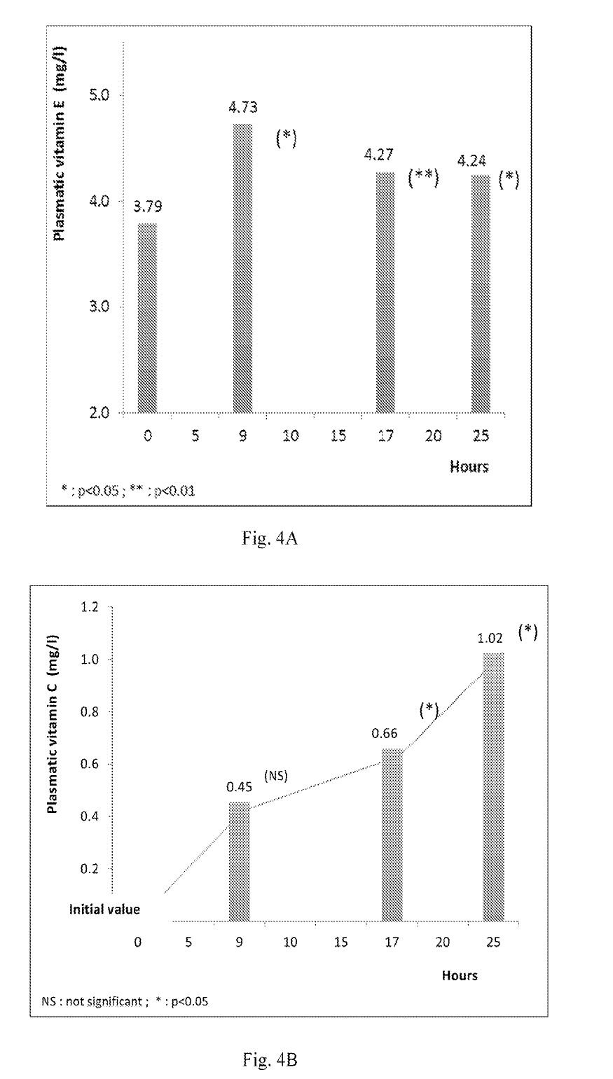

Referring to FIG. 4, the results of this experiment show that the bolus application causes an increase in plasmatic vitamin E at the initial 9 hour measurement, with higher plasmatic vitamin E (compared to t=0) maintained through t=25 h. Plasmatic vitamin C continues to increase at each interval (t=9 h, 17 h, and 25 h) post-application.

### Example 4: Test of Zootechnical Efficacy on Dairy Cows

The effects of administering a multi-section feed bolus on cattle was examined. The objective of the study was to evaluate the effects of providing antioxidants by an oral bolus on blood parameters, somatic cells count (SCC) and milk production.

Holstein dairy cows (n=138) from four dairy farms in Western France, were randomly assigned to a control group (CONTROL, n=65)) and a trial group (TREATED, n=73). Cows in TREATED were administered two boluses according to an exemplary embodiment of the present invention on the first day of the experiment. Each of these bolus included organic zinc, selenium, vitamin E, vitamin C, SOD and aromatic substances.

Milk production, somatic cells count and blood samples were performed before application of the first bolus and then 30 days after application of the boluses. Blood serum was analyzed for vitamin E and vitamin C at the Frank Duncombe's Lab, France.

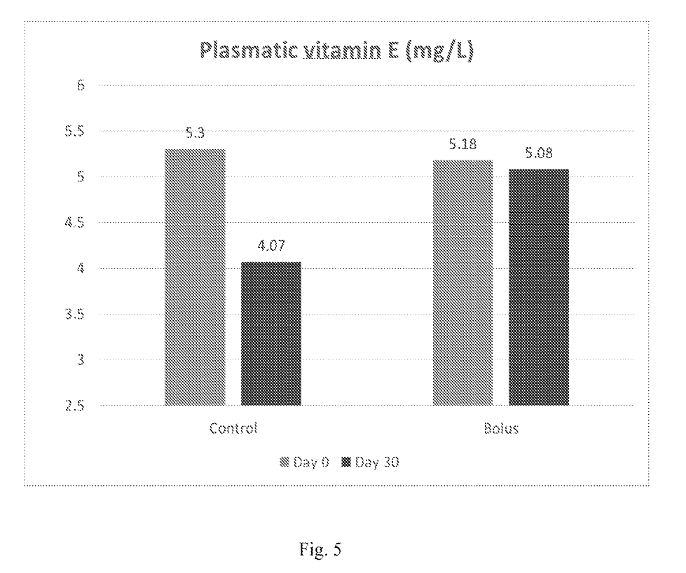

As shown in Table 1, the use of the bolus decreased the overall SCC but its effect was significant for high SCC cows (>300,000 cells/mL). It also had a significant effect on the stabilization of vitamin E level in serum (see FIG. 5). Due to the decrease of somatic cell counts in high risk cows, the bolus significantly increased milk production of 2.5 kg/day.

TABLE 1
Somatic Cells Count

| SCC | | | SCC before | Evolution of | | |
|---|---|---|---|---|---|---|
| classes | | | treatment | SCC | | |
| (×1000 | | | (×1000 | (×1000 | Evolution | P- |
| cells/mL) | Group | n | cells/mL) | cells/mL) | (%) | value |
| 0-100 | Control | 18 | 47.9 | 34.4 | +72% | 0.024 |
| | Treated | 21 | 54.8 | 79.6 | +145% | 0.056 |
| 100-300 | Control | 32 | 186.7 | 150.0 | +80% | 0.423 |
| | Treated | 33 | 185.0 | 120.0 | +65% | 0.205 |
| >300 | Control | 15 | 760.5 | 481.3 | +63% | 0.554 |
| | Treated | 19 | 769.2 | −37.2 | −5% | 0.012 |

>300,000 cells/mL

Control group: SCC increase of 63%

Treated group: SCC decrease of 5% (P<0.05).

### Example 5: Test of Zootechnical Efficacy on Dairy Cows

The effects of administering a multi-section feed bolus on cattle were examined. The objective of this study was to evaluate the effects of providing antioxidants by two applications of an oral bolus on somatic cells count (SCC)

Holstein dairy cows (n=52) at 202 days in milk, from two dairy farms in Western France, were randomly assigned to a control group (CONTROL, n=26) and a trial group (TREATED, n=26). Cows in TREATED were administered two boluses according to an exemplary embodiment of the present invention on the first day (d 0) of the experiment and two boluses at day 30 (d 30). Each of these bolus included: 1) a first, fast-release section including vitamin E, vitamin C, and aromatic substances; and 2) a second, slower-release section including organic zinc, selenium, vitamin E, SOD, and aromatic substances.

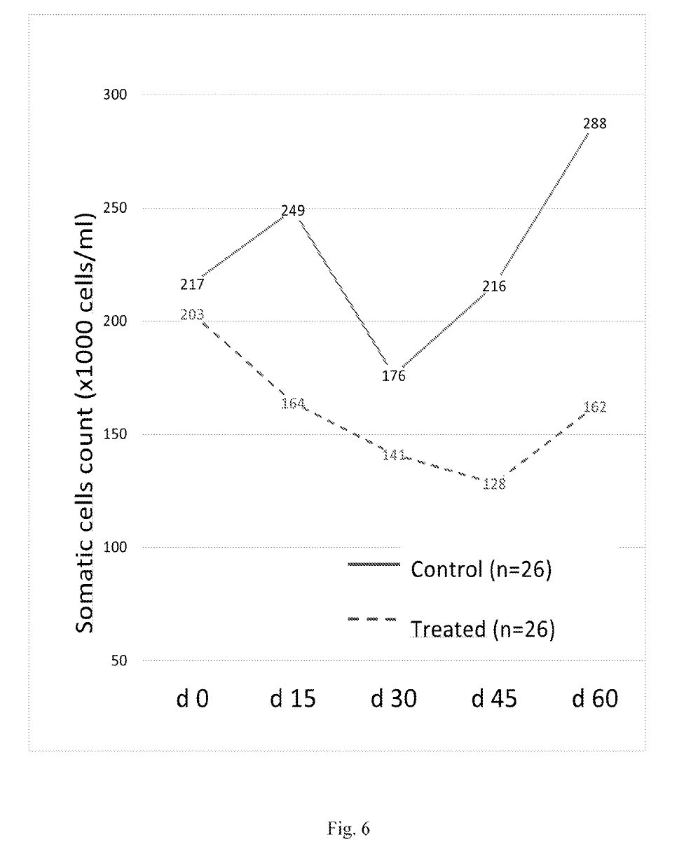

Somatic cells count measurement was performed before the first administration of the boluses, and then every 15 days until d 60 (FIG. 6). As shown in FIG. 6, the use of the bolus decreased the overall SCC at d 60 (162,000 cells in TREATED vs 288,000 in CONTROL).

The effect was significant for high SCC cows (>300,000 cells/mL) (Table 2).

TABLE 2
Somatic Cells Count

| Classes | | | |
|---|---|---|---|
| (×1000 cells/ml) | Group | d 0 | d 60 |
| <100 | Control (n = 12) | 60.4^{a} | 116.4^{a} |
| | Treated (n = 13) | 45.8^{a} | 94.8^{a} |
| 100-300 | Control (n = 10) | 168.2^{a} | 300.3^{a} |
| | Treated (n = 9) | 171.0^{a} | 193.5^{a} |
| >300 | Control (n = 4) | 778.2^{a} | 512.5^{a} |
| | Treated (n = 4) | 830.6^{a} | 250.3^{b} |

### Example 6: Test of Zootechnical Efficacy on Dairy Cows: Milk Production

Holstein dairy cows from one dairy farms in Western France, were randomly assigned to a control group (CONTROL, n=10) and a trial group (TREATED, n=11). Cows in TREATED were administered two boluses 30 days before calving and at calving. Each of these bolus included: 1) a first, fast-release section with vitamin E, vitamin C, and aromatic substances; and 2) a second, slower-release section with organic zinc, selenium, vitamin E, SOD, and aromatic substances.

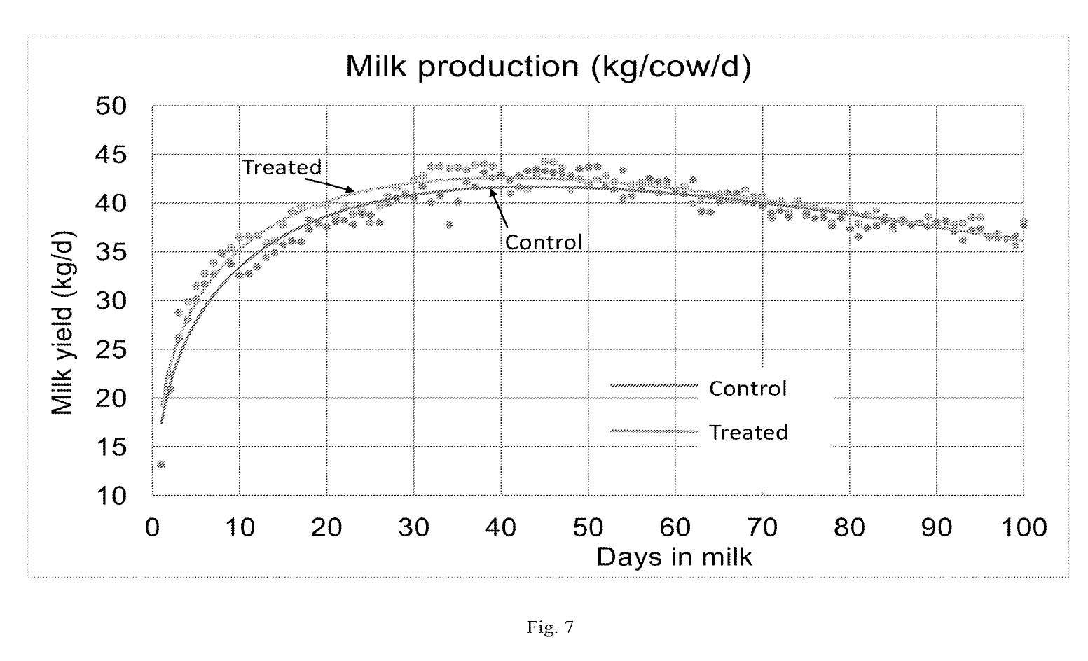

Milk production was performed in the first 100 days of lactation (see FIG. 7). The bolus significantly increased milk production of 0.83 kg/day (39.2 kg/d in TREATED vs. 38.3 kg/d in CONTROL: p=0.005).

The disclosures of each and every patent, patent application, or publication cited herein are hereby incorporated by reference in their entirety. While this invention has been disclosed with reference to specific embodiments, other embodiments and variations of this invention may be devised by others skilled in the art without departing from the true spirit and scope of the invention. The appended claims are intended to be construed to include all such embodiments and variations.

1. An animal feed bolus, comprising:
a first section, and
a second section connected to the first section,
wherein the first section has a different composition and release rate than the second section.

2. (canceled)

3. The bolus of claim 1, wherein the release rate of the first section is less than one day.

4. The bolus of claim 1, wherein the release rate of the first section is less than 12 hours.

5. The bolus of claim 1, wherein the release rate of the first section is less than 6 hours.

6. The bolus of claim 1, wherein the release rate of the first section is less than 1 hour.

7. The bolus of claim 2, wherein the release rate of the second section is greater than 2 days.

8. The bolus of claim 2, wherein the release rate of the second section is greater than 1 week.

9. The bolus of claim 2, wherein the release rate of the second section is greater than 15 days.

10. (canceled)

11. The bolus of claim 1, wherein the first section comprises about 50 wt % of the bolus.

12. The bolus of claim 1, wherein the first section comprises about 40 wt % of the bolus.

13. The bolus of claim 1, wherein the first section comprises less than about 30 wt % of the bolus.

14. The bolus of claim 1, wherein the first section comprises less than about 20 wt % of the bolus.

15. The bolus of claim 1, wherein the first section comprises less than about 10 wt % of the bolus.

16. The bolus of claim 1, wherein the first section comprises vitamin E.

17. (canceled)

18. The bolus of claim 1, wherein the second section comprises a nutrient selected from the group consisting of zinc, selenium, manganese, copper, iodine, and cobalt or salts thereof, oxides thereof, organic forms thereof, and any combination thereof.

19. The bolus of claim 1, wherein the second section comprises one or more aromatic extracts.

20. The bolus of claim 1, wherein the first and/or second section further comprises vitamins A, D3, E, B1, B3, B12, or C, or any combination thereof.

21-31. (canceled)

32. A method for feeding an animal, comprising:
providing a bolus having a first section and a second section, wherein the first section is connected to the second section and the first and second sections have different release rates, and
administering the bolus to the animal.

33. The method of claim 32, wherein the animal is a ruminant.

34. The method of claim 32, wherein the bolus remains in the reticulum of the ruminant after administration.

35. (canceled)
